(19) United States
(12) Patent Application Publication
(10) Pub. No.: US 2026/0224409 A1
(43) Pub. Date: Aug. 6, 2026

(54) ABSORBENT ARTICLE

(71) Applicant: LIVEDO CORPORATION, Ehime (JP)

(72) Inventors: Yoshiko IWAMOTO, Tokushima (JP); Yuki TAKAHASHI, Tokushima (JP)

(73) Assignee: LIVEDO CORPORATION, Ehime (JP)

(21) Appl. No.: 19/453,740

(22) Filed: Jan. 20, 2026

(30) Foreign Application Priority Data
Jan. 31, 2025 (JP) 2025-015569

(51) Int. Cl. A61F 13/15; A61F 13/511

(52) U.S. Cl. CPC A61F 13/15203; A61F 13/511; A61F 2013/15121; A61F 2013/15357; A61F 2013/15382; A61F 2013/15878

(57) ABSTRACT

An absorbent article (1) includes a top sheet (2), a back sheet (3) and an absorbent body (4) disposed therebetween. The top sheet (2) includes thick parts (21) and thin parts (22) that extend in parallel in the front-rear direction and are arranged alternately in the width direction. The thick parts (21) and the thin parts (22) extend from a portion overlapping with the absorbent body (4) to positions forward beyond a front edge (41) of the absorbent body (4) and to positions rearward beyond a rear edge (42) of the absorbent body (4). The top sheet has a skin-facing surface (23) and a non-skin-facing surface (24) with the thin parts (22) that are recessed relative to the thick parts (21) on both the skin-facing and the non-skin-facing surfaces (23)(24). The thickness of the thick part (21) is 3 to 15 times the thickness of the thin part (22).

### DESCRIPTION

This application is related to and claims priority under 35 U.S.C. 119 to Japanese patent application No. 2025-015569, filed on January 31, 2025.

### TECHNICAL FIELD

The present invention relates to an absorbent article such as urine pads (including light incontinence pads), sanitary napkins and disposable diapers.

### BACKGROUND OF THE INVENTION

Various top sheets used in absorbent articles are known. For example, Patent Document 1 discloses a nonwoven fabric having ridges and grooves extending in parallel in the front-rear direction and being alternately arranged in the width direction, and Patent Document 2 discloses an absorbent article including a nonwoven fabric as a top sheet having ridges and grooves alternately arranged in the width direction.

### CITATION LIST

### Patent Document

### Patent Document 1

Japanese Unexamined Laid-open Patent Application Publication No. 2010-285735

### Patent Document 2

Japanese Unexamined Laid-open Patent Application Publication No. 2017-038838

### SUMMARY OF INVENTION

### Technical Problem

In general, absorbent articles are formed to have front and rear end portions where an absorbent body is absent, and thus, the front and rear end portions tend to have lower shape retention than a portion where the absorbent body is present. Accordingly, when a user handles the absorbent article by grasping the front and rear end portions, the absorbent article tends to bend at front and rear edges of the absorbent body, which may reduce the handleability of the absorbent article. The present invention has been made in view of the above circumstances, and an object of the present invention is to provide an absorbent article that is less likely to bend at the front and rear edges of the absorbent body when handled by grasping the front and rear end portions of the absorbent article.

### Solution to Problem

The absorbent article of the present invention, which achieves the above object, is as follows.

[1] An absorbent article has a front-rear direction and a width direction, and includes a top sheet, a back sheet and an absorbent body disposed between the top sheet and the back sheet. The top sheet includes thick parts and thin parts that extend in parallel in the front-rear direction and are arranged alternately in the width direction. The thick parts and the thin parts extend from a portion overlapping with the absorbent body to positions forward beyond a front edge of the absorbent body and to positions rearward beyond a rear edge of the absorbent body. The top sheet has a skin-facing surface with the thin parts that are recessed relative to the thick parts and a non-skin-facing surface with the thin parts that are recessed relative to the thick parts. The thickness of the thick part is 3 times or more and 15 times or less the thickness of the thin part.

[2] The absorbent article according to [1] above, wherein the top sheet is a nonwoven fabric that is heat-embossed at the thin parts.

[3] The absorbent article according to [2] above, wherein the nonwoven fabric includes a fused portion of fibers in a vicinity of a boundary of the thick part and the thin part, a ratio of a long fused portion, in which a fusion length between fibers is 10 times or more a diameter of the fiber, is less than 5%, and a total ratio of a medium fused portion, in which the fusion length of fibers is 3 times or more and less than 10 times the diameter of the fiber, and a short fused portion, in which the fusion length of fibers is 1.5 times or more and less than 3 times the diameter of the fiber, is 20% or more and 100% or less.

[4] The absorbent article according to any one of [1] to [3] above, wherein the mass per unit area of the top sheet is 20 g/m² or more and 35 g/m² or less.

[5] The absorbent article according to any one of [1] to [4] above, wherein the thick parts and the thin parts extend continuously in the front-rear direction from a front edge to a rear edge of the top sheet.

[6] The absorbent article according to any one of [1] to [5] above, wherein the front edge of the absorbent body is straight and the rear edge of the absorbent body is straight.

[7] The absorbent article according to any one of [1] to [6] above, wherein the top sheet includes a portion that is positioned forward of the front edge of the absorbent body and the portion has a length, that is 2 cm or longer and 7 cm or shorter, in the front-rear direction, and the top sheet includes a portion that is positioned rearward of the rear edge of the absorbent body and the portion has a length, that is 2 cm or longer and 7 cm or shorter, in the front-rear direction.

### Advantageous Effects of Invention

In the absorbent article of the present invention, shape retention of the top sheet in the front-rear direction is enhanced in the area, where thick parts and thin parts are present, of the top sheet. This area with enhanced shape retention extends from the portion overlapping with the absorbent body to positions forward beyond the front edge of the absorbent body and rearward beyond the rear edge of the absorbent body. With this configuration, the absorbent article becomes less likely to bend at the front and rear edges of the absorbent body. The thin parts of the top sheet are recessed relative to the thick parts on both the skin-facing surface and the non-skin-facing surface of the top sheet, and the thickness of each of the thick parts is configured to be 3 times or more and 15 times or less the thickness of each of the thin parts. As a result, the top sheet tends to become less likely to bend unintentionally in the front-rear direction on both the skin-facing surface and the non-skin-facing surface. With this configuration, the absorbent article tends to be maintained in a flat state when attached to underwear or set on a wearer's crotch, resulting in better handleability of the absorbent article. Furthermore, when wearing the absorbent article, it is easier to properly handle the absorbent article without grasping at the portion where the absorbent body is present. As a result, the possibility of damaging the absorbent body is reduced.

### BRIEF DESCRIPTION OF THE DRAWINGS

FIG. 1 illustrates a plan view of a light incontinence pad, which is an example of an absorbent article of the present invention, as seen from a top sheet side thereof.

FIG. 2 illustrates a widthwise cross-sectional view of a top sheet included in the absorbent article shown in FIG. 1.

FIG. 3 illustrates a cross-sectional view taken along a line III-III of the absorbent article shown in FIG. 1.

FIG. 4 illustrates a schematic plan view of an example of an adhesive coating arrangement with adhesive regions on a non-skin-facing surface of the top sheet in the absorbent article shown in FIG. 1.

FIG. 5 illustrates an example of an absorbent body included in the absorbent article shown in FIG. 1, and shows a widthwise cross-sectional view of the absorbent body.

FIG. 6 illustrates a partial cutaway plan view of the absorbent body shown in FIG. 5 as seen from a top sheet side thereof.

### DETAILED DESCRIPTION OF EMBODIMENTS

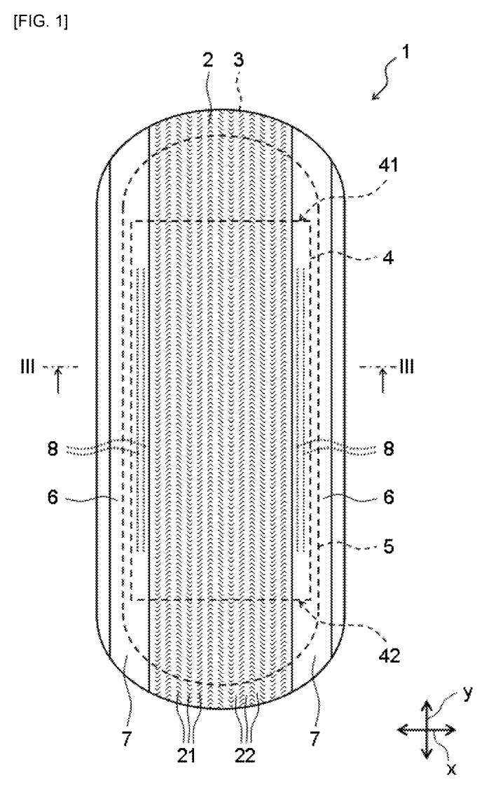

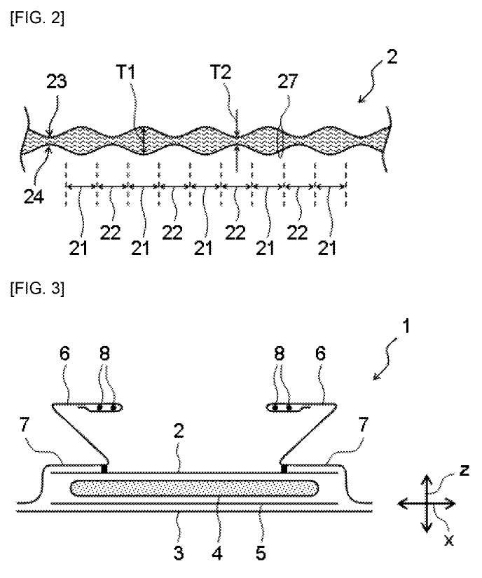

The example of an absorbent article of the present invention is explained with reference to drawings. However, the present invention is not limited to embodiments shown in the drawings. FIGS. 1 to 3 show a configuration example of a light incontinence pad as an embodiment of an absorbent article of the present invention. As used herein, the singular forms “a,” “an,” and “the” include plural referents unless the context clearly dictates otherwise. FIG. 1 shows a plan view of the absorbent article (a light incontinence pad) as seen from a top sheet side thereof. FIG. 2 shows a widthwise cross-sectional view of a top sheet included in the absorbent article shown in FIG. 1. FIG. 3 shows a cross-sectional view taken along a line III-III of the absorbent article shown in FIG. 1. The absorbent articles according to the present invention are not limited to the embodiments shown in the drawings.

An absorbent article 1 includes a top sheet 2, a back sheet 3 and an absorbent body 4 disposed between the top sheet 2 and the back sheet 3. The top sheet 2 is disposed at a side closer to a skin-facing surface of the absorbent body 4, and the back sheet 3 is disposed at a side closer to a non-skin-facing surface of the absorbent body 4. Excrement that has passed through the top sheet 2 is absorbed into the absorbent body 4. The back sheet 3 prevents excrement from leaking to the outside. Embodiments of the absorbent article 1 can be applied to, for example, urine pads (including light incontinence pads), sanitary napkins, disposable diapers, and the like, and are particularly suitable for light incontinence pads and sanitary napkins.

The absorbent article 1 has a front-rear direction y and a width direction x. The front-rear direction y indicates a direction extending in a front-rear direction at a crotch of a wearer when the absorbent article is worn. The width direction x indicates a direction orthogonal to the front-rear direction y on the same plane as the absorbent article 1. The width direction x corresponds to a left-right direction of a wearer when the absorbent article is worn. Furthermore, a direction parallel to the plane defined by the front-rear direction y and the width direction x is referred to as a planar direction, and a direction orthogonal to this plane is referred to as a thickness direction z. The absorbent article 1 has a skin-facing surface and a non-skin-facing surface that are defined in the thickness direction z.

The top sheet 2 is provided so as to face a wearer’s skin when the absorbent article 1 is worn. The top sheet 2 is preferably liquid permeable. As the top sheet 2, a nonwoven fabric made from hydrophilic fibers such as cellulose, rayon and cotton; a nonwoven fabric which is formed from hydrophobic fibers such as polyolefin (e.g., polypropylene or polyethylene), polyester (e.g., PET) and polyamide (e.g., nylon), and in which the hydrophobic fibers are hydrophilized with a surfactant on the surface thereof; or the like can be used, for example.

The back sheet 3 is preferably liquid impermeable. As the back sheet 3, a nonwoven fabric made from hydrophobic fibers such as polyolefin (e.g., polypropylene or polyethylene), polyester (e.g., PET) and polyamide (e.g., nylon); a plastic film; or the like can be used, for example. Also, as the back sheet 3, a laminate of a nonwoven fabric and a plastic film may be used.

The top sheet 2 and back sheet 3 are preferably longer than the absorbent body 4 in the front-rear direction y. Specifically, the top sheet 2 and the back sheet 3 are preferably arranged to extend forward beyond a front edge 41 and extend rearward beyond a rear edge 42 of the absorbent body 4.

The absorbent article 1 may include a base sheet 5 provided between the absorbent body 4 and the back sheet 3. Providing the base sheet 5 may improve shape retention of the absorbent article 1.

The absorbent article 1 preferably includes standing flaps 6 provided on both sides in the width direction x of the skin-facing surface of the top sheet 2. By providing the standing flap 6, lateral leakage of urine and the like is prevented. The standing flaps can be configured, for example, by adhering side sheets 7 that extend in the front-rear direction y to both sides in the width direction x of the top sheet 2, and providing elastic members 8 for the standing flaps on an inner portion of the side sheets 7 in the width direction x. By providing the side sheets 7 and the elastic members 8 for the standing flaps in this manner, the inner portions of the side sheets 7 in the width direction x stand up toward a wearer's skin due to contraction force of the elastic members 8, thereby forming the standing flaps 6. The standing flaps 6 or the side sheets 7 may be made of a material preferably selected from liquid-impermeable plastic films, liquid-impermeable nonwoven fabrics and a combination selected among the liquid-impermeable plastic films, and the liquid-impermeable nonwoven fabrics.

As an nonwoven fabrics used for a sheet described above, a spunbond nonwoven fabric, a pointbond nonwoven fabric, an air-through nonwoven fabric, a meltblown nonwoven fabric, an airlaid nonwoven fabric, a spunlace nonwoven fabric, an SMS nonwoven fabric, or the like can be used. The mass per unit area of each of these sheets is preferably 8 g/m² or more, more preferably 10 g/m² or more, and preferably 40 g/m² or less.

The absorbent body 4 is not particularly limited as long as it contains an absorbent material that is able to absorb bodily exudates such as urine. As the absorbent body 4, a shaped product of an absorbent material, which is formed into a certain shape, may be used, or the shaped product wrapped with a cover sheet selectable among paper (e.g., tissue paper and thin paper) and liquid-permeable nonwoven fabrics may be used. Examples of the absorbent material include, for example, a hydrophilic fiber such as a cellulose fiber, and an absorbent polymer such as a polyacrylic absorbent polymer, a polyasparaginic absorbent polymer, a cellulosic absorbent polymer and a starch-acrylonitrile absorbent polymer. The absorbent material may include a thermally fusible fiber such as a polyolefin (e.g., polyethylene and polypropylene) fiber, a polyester (e.g., PET) fiber and a polyamide fiber. These thermally fusible fibers may be hydrophilized with a surfactant or the like to increase affinity with urine and the like.

The absorbent material preferably includes a hydrophilic fiber in view of increasing absorption rate of urine and the like. In addition, in view of enhancing absorption capacity, the absorbent material preferably includes an absorbent polymer. Therefore, as one example of the absorbent body 4, it may contain a hydrophilic fiber (especially a pulp fiber) and an absorbent polymer. In this case, the absorbent material to be used is preferably formed by mixing absorbent polymer particles with a hydrophilic-fiber assembly, or dispersing absorbent polymer particles on a hydrophilic-fiber assembly, for example. The absorbent body 4 may include two or more absorbent layers.

The absorbent body 4 may be a sheet-shaped absorbent body. Examples of the sheet-shaped absorbent body includes one that is configured to contain an absorbent polymer but not contain a pulp fiber between sheet members. Such a sheet-shaped absorbent body can achieve a high absorption capacity since it contains an absorbent polymer between sheet members. Furthermore, since the sheet-shaped absorbent body does not contain a pulp fiber between sheet members, it can be formed thin and with reduced bulk.

The absorbent body 4 may include two or more sheet-shaped absorbent layers. For example, the absorbent body 4 may include a first sheet-shaped absorbent layer on a side closer to the top sheet 2 and a second sheet-shaped absorbent layer on a side closer to the back sheet 3. The number of absorbent layers included in the absorbent body 4 may be appropriately determined according to the application, and for example, it may have three or more sheet-shaped absorbent layers. Such an absorbent body 4 can provide an absorbent article with a higher absorption capacity while maintaining a thin profile, since the absorbent polymer is arranged in layers in the thickness direction as well as the planar direction.

A shape (planar shape) of the absorbent body 4 is not particularly limited. The shape of the absorbent body 4 may be appropriately determined according to application, and examples of that include a substantially rectangular shape, an hourglass shape, a center nipped-in gourd shape, a battledore shape and others. In the case where the absorbent body 4 includes two or more absorbent layers, the shapes (planar shapes) and/or sizes of the absorbent layers may differ from each other.

The top sheet 2 includes thick parts 21 and the thin parts 22 that extend in parallel in the front-rear direction y and are alternately arranged in the width direction x. As shown in FIG. 2, the thin parts 22 are recessed relative to the thick parts 21 on a skin-facing surface 23 of the top sheet 2, and the thin parts 22 are recessed relative to the thick parts 21 on a non-skin-facing surface 24 of the top sheet 2. In the top sheet 2, the thickness T1 of the thick part 21 is 3 times or more and 15 times or less the thickness T2 of the thin part 22. The thick parts 21 and the thin parts 22 extend from a portion overlapping with the absorbent body 4 to positions forward beyond the front edge 41 of the absorbent body 4 and to positions rearward beyond the rear edge 42 of the absorbent body 4. With this configuration of the top sheet 2, the absorbent article 1 is less likely to bend unintentionally at the front edge 41 and the rear edge 42 of the absorbent body 4 when a user handles the absorbent article 1 by grasping the front end part and the rear end part, where the absorbent body 4 is not present, of the absorbent article 1. That is, since in the area where the thick parts 21 and the thin parts 22 of the top sheet 2 are present, the shape retention of the top sheet 2 in the front-rear direction is enhanced and this enhanced shape retention area extends from the portion overlapping with the absorbent body 4 to the positions forward beyond the front edge 41 of the absorbent body 4 and to rearward beyond the rear edge 42 of the absorbent body 4. Accordingly, the absorbent article 1 becomes less likely to bend unintentionally at the front edge 41 and the rear edge 42 of the absorbent body 4. This improves the handling of the absorbent article 1 when attaching it to underwear or placing it on a wearer's crotch. Furthermore, when wearing the absorbent article 1, it becomes easier to properly handle the absorbent article 1 without grasping the portion where the absorbent body 4 is present, thereby reducing the possibility of damaging the absorbent body 4. The portion where the top sheet 2 overlaps with the absorbent body 4 and the regions where the top sheet 2 does not overlap with the absorbent body 4 are determined in a plan view of the absorbent article 1.

In the top sheet 2, the thick parts 21 are configured to be thicker than the thin parts 22. The thick parts 21 are configured to protrude more than the thin parts 22 on both the skin-facing surface 23 and the non-skin-facing surface 24 of the top sheet 2; that is, on the skin-facing surface 23 of the top sheet 2, the thick parts 21 are configured to protrude towards a skin-facing side relative to the thin parts 22, and on the non-skin-facing surface 24 of the top sheet 2, the thick parts 21 are configured to protrude towards a non-skin-facing side relative to the thin parts 22. On the top sheet 2, the thick parts 21 and the thin parts 22 extend in parallel in the front-rear direction y and are alternately arranged in the width direction x. The thick parts 21 and the thin parts 22 are arranged in a stripe pattern extending in the front-rear direction y. With this configuration, the top sheet 2 becomes less likely to bend unintentionally in the front-rear direction y toward a skin-facing side and a non-skin-facing side, and thus, the absorbent article 1 tends to be maintained in a flat state when attached to underwear or set on a wearer's crotch. Furthermore, since the thickness T1 of the thick part 21 is preferably 3 times or more the thickness T2 of the thin part 22, the difference between the thickness T1 of the thick part 21 and the thickness T2 of the thin part 22 becomes large, resulting in the top sheet 2 becoming less likely to bend unintentionally in the front-rear direction y. Also, since the thickness T1 of the thick part 21 is 15 times or less the thickness T2 of the thin part 22, the thickness at the thin part 22 is sufficiently ensured, resulting in the top sheet 2 becoming less likely to bend unintentionally in the width direction x. The thickness T1 of the thick part 21 is preferably 12 times or less, more preferably 10 times or less the thickness T2 of the thin part 22.

Furthermore, with the configuration and arrangement of the thick parts 21 and the thin parts 22 of the top sheet 2 as described above, the diffusion of urine and the like in the front-rear direction y on the skin-facing surface 23 of the top sheet 2 and the diffusion of urine and the like in the front-rear direction y in gaps between the top sheet 2 and the absorbent body 4 become enhanced. Since the thin parts 22 are recessed relative to the thick parts 21 on the skin-facing surface 23 of the top sheet 2, the thin parts 22 are expected to become diffusion paths for urine and the like in the front-rear direction y. Accordingly, diffusibility of urine and the like in the front-rear direction y is enhanced. Meanwhile, since the thin parts 22 are recessed relative to the thick parts 21 on the non-skin-facing surface 24 of the top sheet 2, the thin parts 22 at a side adjacent to the non-skin-facing surface of the top sheet 2 are expected to become diffusion gaps for urine and the like. Accordingly, diffusibility of urine and the like in the front-rear direction y in the gaps is enhanced.

The thickness T1 of the thick part 21 and the thickness T2 of the thin part 22 of the top sheet 2 can be determined by peeling the top sheet 2 from the absorbent article 1, cutting the top sheet along the width direction x, and measuring the thickness T1 of the thick part 21 and the thickness T2 of the thin part 22 at the cut surface. The thickness T1 of the thick part 21 is determined by measuring the thickness at the thickest part of the thick part 21 located between adjacent thin parts 22 in the width direction x. The thickness T2 of the thin part 22 is determined by measuring the thickness at the thinnest part of the thin part 22 located between adjacent thick parts 21 in the width direction x. Cutting of the top sheet 2 along the width direction x is performed at three positions: a first position that is within 1 cm forward of the front edge 41 of the absorbent body 4, a second position that is within 1 cm rearward of the rear edge 42 of the absorbent body 4, and a third position that is a center position in the front-rear direction y and at the center position the top sheet 2 overlaps with the absorbent body 4; the thicknesses of five or six thick parts 21 and the thin parts 22 that are positioned in a central portion in the width direction x are measured at each of these three positions; and their average values are calculated to obtain the thickness T1 of the thick part 21 and the thickness T2 of the thin part 22. The thicknesses of the thick part 21 and the thin part 22 can be conveniently measured from an image of the cut surface of the top sheet 2.

The top sheet 2 is preferably a nonwoven fabric, and the thick parts 21 and the thin parts 22 are preferably formed by embossing the nonwoven fabric to thin the nonwoven fabric at the embossed portions. That is, in the top sheet 2, the thin parts 22 are preferably the embossed portions. This ensures shape retention in the thin parts 22 of the top sheet 2, which makes the top sheet 2 less likely to bend unintentionally in the front-rear direction y and also less likely to bend unintentionally in the width direction x at the thin part 22. The embossed portion of the thin part 22 may be a heat-embossed portion (i.e., a portion embossed with heating) or a non-heat-embossed portion (i.e., a portion embossed without heating), and the former heat-embossed portion is more preferred. That is, it is more preferred that the nonwoven fabric is heat-embossed at the thin part 22 of the top sheet 2. This makes it easier to ensure shape retention at the thin parts 22 of the top sheet 2.

To form the thin parts 22 recessed relative to the thick parts 21 on both the skin-facing surface 23 and the non-skin-facing surface 24 of the top sheet 2, as one example, an embossing roll having ridges and grooves circumferentially extending in parallel and that are arranged alternately in the axial direction on its circumferential surface and a flat roll may be prepared, and a nonwoven fabric may be sandwiched between the embossing roll and the flat roll to be embossed. As another example, two embossing rolls may be prepared, each having ridges and grooves that circumferentially extend in parallel and that are arranged alternately in the axial direction on each circumferential surface, The two embossing rolls may be arranged with the ridges of the two embossing rolls facing each other, and a nonwoven fabric may be sandwiched between the two embossing rolls to be embossed. In the case where the thin parts 22 are heat-embossed portions, one or both of the two rolls may be heated rolls. In the above example, it is preferable to use the two rolls that are heated.

The thickness T1 of the thick part 21 of the top sheet 2 is preferably 0.3 mm or more, more preferably 0.4 mm or more. This configuration ensures the thickness of the top sheet 2, and the top sheet 2 becomes less likely to bend unintentionally in the front-rear direction y. Meanwhile, the thickness T1 of the thick part 21 of the top sheet 2 is preferably 1.8 mm or less, more preferably 1.5 mm or less. This configuration prevents the top sheet 2 from becoming excessively thick, and the liquid permeability of the top sheet 2 is easily ensured.

The arrangement pitch of the thick parts 21 that are arranged side by side in the width direction x is preferably 1 mm or more, more preferably 2 mm or more, and preferably less than 5 mm. The arrangement pitch of the thick parts 21 means a distance in the width direction x between the thickest part of a first thick part 21 and the thickest part of a second thick part 21 that is adjacent to the first thick part 21. With this configuration, the effect of the top sheet 2 including the thick parts 21 and the thin parts 22 becoming less likely to bend unintentionally in the front-rear direction y can be readily achieved. The arrangement pitch of the thin parts 22 that are arranged side by side in the width direction x may be the same as the arrangement pitch of the thick parts 21 described above.

In the top sheet 2, the thick parts 21 and the thin parts 22 described above are preferably arranged over a larger area that is forward of the front edge 41 of the absorbent body 4 and preferably arranged over a larger area that is rearward of the rear edge 42 of the absorbent body 4. In the portion forward of the front edge 41 of the absorbent body 4 in the top sheet 2, the lengths (maximum lengths) in the front-rear direction y of the thick parts 21 and the thin parts 22 arranged in a striped pattern are preferably 50% or longer, more preferably 70% or longer, even preferably 80% or longer of the length in the front-rear direction y of that area. It is particularly preferable that the thick parts 21 and the thin parts 22 arranged in a stripe pattern extend in parallel entirely in the front-rear direction y in the area forward of the front edge 41 of the absorbent body 4 of the top sheet 2. In the area rearward of the rear edge 42 of the absorbent body 4 of the top sheet 2, the lengths in the front-rear direction y of the thick parts 21 and the thin parts 22 arranged in a stripe pattern are preferably 50% or longer, more preferably 70% or longer, even preferably 80% or longer of the length in the front-rear direction y of that area. It is particularly preferable that the thick parts 21 and the thin parts 22 arranged in a stripe pattern extend entirely in the front-rear direction y in the area rearward of the rear edge 42 of the absorbent body 4 of the top sheet 2. It is particularly preferable that the thick parts 21 and the thin parts 22, that are arranged in a stripe pattern, extend in parallel in the front-rear direction y and are arranged alternately in the width direction x in the area rearward of the rear edge 42 of the absorbent body 4 of the top sheet 2.

In the top sheet 2, it is preferable that the thick parts 21 and the thin parts 22 arranged in a stripe pattern are provided over a larger area that overlaps with the absorbent body 4. In the portion, that overlaps with the absorbent body 4, of the top sheet 2, the lengths (maximum lengths) in the front-rear direction y of the thick parts 21 and the thin parts 22 arranged in a stripe pattern are preferably 20% or longer, more preferably 40% or longer, even more preferably 60% or longer, and still even more preferably 80% or longer of the length in the front-rear direction y of the absorbent body 4. It is particularly preferable that the thick part 21 and the thin parts 22 extends continuously in the front-rear direction y from the front edge 41 to the rear edge 42 of the absorbent body 4.

It is preferable that the thick parts 21 and the thin parts 22 extend continuously in the front-rear direction y from a front edge to a rear edge of the top sheet 2. With this configuration, the absorbent article 1 becomes less likely to bend unintentionally over the entire length in the front-rear direction y.

The entire width in the width direction x of the stripe pattern in that the thick parts 21 and the thin parts 22 are alternately arranged is preferably 80% or wider, more preferably 90% or wider of the width in the width direction x of the absorbent body 4, and may even be wider than the width in the width direction x of the absorbent body 4. In addition, the entire width in the width direction x of the stripe pattern in that the thick parts 21 and the thin parts 22 are alternately arranged is preferably 80% or wider, more preferably 90% or wider of the width in the width direction x of the top sheet 2. With this configuration of the absorbent article 1, shape retention tends to be ensured in the planar direction over a larger range in the width direction x of the absorbent article 1.

The mass per unit area of the top sheet 2 is preferably 20 g/m² or more and 35 g/m² or less. With this configuration of the top sheet 2, the thick parts 21 and the thin parts 22 are easily formed such that the thickness T1 of the thick part 21 is 3 times or more and 15 times or less the thickness T2 of the thin part 22 by embossing the nonwoven fabric of the top sheet 2. The mass per unit area of the top sheet 2 is preferably 22 g/m² or more, more preferably 25 g/m² or more, and preferably 32 g/m² or less, more preferably 30 g/m² or less. The thickness T1 of the thick part 21 and the thickness T2 of the thin part 22 can also be adjusted by selecting among nonwoven fabric types, changing embossing pressure, changing heat-embossing temperature, and the like.

It is preferable that the thick part 21 of the top sheet 2 includes a fused portion in which fibers of the nonwoven fabric are fusion-bonded to each other in the vicinity of a boundary with the thin part 22. Hereinafter, the vicinity of the boundary, that is in the thick part 21, with the thin part 22 may be referred to as the vicinity of the boundary between the thick part 21 and the thin part 22. In FIG. 2, the vicinity of the boundary between the thick part 21 and the thin part 22 is shown as being surrounded by an ellipse denoted by reference numeral 27.

As described above, the top sheet 2 is preferably a nonwoven fabric that is heat-embossed at the thin parts 22. In this case, it is preferable that there is a fused portion, where fibers of the nonwoven fabric of the top sheet 2 are fusion-bonded to each other, in the vicinity of the boundary between the thick part 21 and the thin part 22. With this configuration the thick parts 21 become less likely to be flattened, and the thickness of the thick part 21 becomes easier to be maintained. That is, at a part in the vicinity of the boundary between the thick part 21 and the thin part 22, the skin-facing surface 23 and non-skin-facing surface 24 of the top sheet 2 tend to extend obliquely with respect to the thickness direction z in the widthwise cross-section of the top sheet 2, and when the fused portion is included in this part, the shape of this part of the top sheet 2 tends to be more easily maintained.

In the vicinity of the boundary between the thick part 21 and the thin part 22, it is preferable that the ratio of a long fused portion, in which the fusion length between fibers is 10 times or more the diameter of the fiber, among the fused portions is less than 5%. When the thin parts 22 are formed by heat-embossing, liquid permeability in the thin parts 22 may decrease in the top sheet 2, so it is desirable to ensure liquid permeability in the thick parts 21. In this respect, when the ratio of the long fused portion among the fused portions in the vicinity of the boundary between the thick part 21 and the thin part 22 is less than 5%, it becomes easier to ensure liquid permeability in the thick part 21. The ratio of the long fused portion among the fused portions in the vicinity of the boundary between the thick part 21 and the thin part 22 may be less than 3% or less than 1%, or it is preferable that the vicinity of the boundary between the thick part 21 and the thin part 22 does not include a long fused portion.

In the vicinity of the boundary between the thick part 21 and the thin part 22, the total ratio of a medium fused portion, in which the fusion length between fibers is 3 times or more and less than 10 times the diameter of the fiber, and a short fused portion, in which the fusion length between fibers is 1.5 times or more and less than 3 times the diameter of the fiber, among the fused portions is preferably 20% or more and 100% or less. In the vicinity of the boundary between the thick part 21 and the thin part 22, the number of the short fused portions is preferably greater than the number of the medium fused portions among the above fused portions, as this allows texture of the nonwoven fabric of the top sheet 2 to be maintained. Furthermore, it is preferable that the vicinity of the boundary between the thick part 21 and the thin part 22 does not include a long fused portion to maintain the texture of the nonwoven fabric of the top sheet 2.

The vicinity of the boundary between the thick part 21 and the thin part 22 can be defined as follows. In the case that the boundary between the thick part 21 and the thin part 22 is clearly defined, the range in the thick part 21 that is 0.0W to 0.1W away from the boundary between the thick part 21 and the thin part 22, where W is the width of the thick part 21 in the width direction x, can be defined as the vicinity of the boundary between the thick part 21 and the thin part 22. On the other hand, in the case that the boundary between the thick part 21 and the thin part 22 is unclear as shown in FIG. 2, a range extending from 0.4D to 0.5D away from the thickest part of the thick part 21, where D is the distance in the width direction x between the thickest part of the thick part 21 and the thinnest part of the thin part 22 that is adjacent to the thick part 21, can be defined as the vicinity of the boundary between the thick part 21 and the thin part 22. By observing the skin-facing surface of the thick part 21 from the thin part 22 side with an electron microscope, the presence or absence of a fused portion, the ratio of the long fused portions, the ratio of the medium fused portions, and the ratio of the short fused portions among the fused portions can be determined. The specific procedure is as follows.

The top sheet 2 is peeled off from the absorbent article 1 and removed, and the top sheet 2 that is removed from the absorbent article 1 is cut along a cutting line extending in the front-rear direction y along the thickest part of the thick part 21, cut along a cutting line extending in the front-rear direction y along the thinnest part of the adjacent thin part 22, and further cut along a cutting line extending in the width direction x. A cross section of the top sheet 2 cut along the cutting line extending in the width direction x is subjected to gold sputter-coating (the deposition time for coating is set to the minimum time necessary for observation), and then the vicinity of the boundary between the thick part 21 and the thin part 22 in the cross section is observed using a scanning electron microscope (SEM). The observation magnification is set to 50 times or more and 300 times or less, depending on the fiber diameter. In the observation area, the portions where fibers are fusion-bonded to each other at their fiber intersections are marked, and the fusion length of each fused portion is measured. The total number of fused portions, the number of the long fused portion where the fusion length is 10times or more the diameter of the fiber, the number of the medium fused portion where the fusion length is 3 times or more and less than 10 times the diameter of the fiber, and the number of the short fused portion where the fusion length is 1.5 times or more and less than 3 times the diameter of the fiber are counted, and the ratio of each fused portion is calculated using the total number of fused portions as the denominator. Similarly, 10 different locations are observed to determine the ratio of each fused portion, and the average of these values is taken as the ratio of each fused portion. To determine whether a portion is fused, the fiber shape is compared with that of an unfused portion, and a portion that appears to be fused with fiber resin and has a different shape at the fiber intersection from that of an unfused portion is considered to be the fused portion. Furthermore, if the fiber diameters of two fibers in the fused portion are different, the average of the diameters of the two fibers is considered to be the diameter of the fiber in the fused portion.

From the viewpoint that the effect of forming the thick parts 21 and the thin parts 22 in the top sheet 2 as described above is more effectively achieved, the absorbent article 1 is preferably has the absorbent body 4 with at least one of the front edge 41 and rear edge 42 of the absorbent body 4 being straight, and more preferably both the front edge 41 and rear edge 42 of the absorbent body 4 are straight. When the front edge 41 and rear edge 42 of the absorbent body 4 are f straight, the absorbent article 1 tends to be easily bent at the edges; however, the thick parts 21 and the thin parts 22 are arranged on the top sheet 2 to extend in parallel in the front-rear direction y in a stripe pattern, the effect of the absorbent article 1 becoming less likely to bend unintentionally at the front edge 41 and the rear edge 42 of the absorbent body 4 is more readily achieved. In this case, the front edge 41 and the rear edge 42 of the absorbent body 4 are preferably formed to be straight to extend in the width direction x. An example of such an absorbent body 4 is a rectangular-shaped absorbent body 4.

The length in the front-rear direction y of the portion, that is positioned forward of the front edge 41 of the absorbent body 4, of the top sheet 2 is preferably 2 cm or longer and 7 cm or shorter. The length in the front-rear direction y of the portion, that is positioned rearward of the rear edge 42 of the absorbent body 4, of the top sheet 2 is preferably 2 cm or longer and 7 cm or shorter. With this configuration of the absorbent article 1, the effect of arrangement of the top sheet 2 on that the thick parts 21 and the thin parts 22 extend in parallel in the front-rear direction y in a stripe pattern, namely, the effect of the absorbent article 1 becoming less likely to bend unintentionally at the front edge 41 and the rear edge 42 of the absorbent body 4, is more reliably achieved. The length in the front-rear direction y of the portion, that is positioned forward of the front edge 41 of the absorbent body 4, is more preferably 2.5 cm or longer, and more preferably 6 cm or shorter, and the length in the front-rear direction y of the portion that is positioned rearward of the rear edge 42 of the absorbent body 4 is more preferably 2.5 cm or longer, and more preferably 6 cm or shorter.

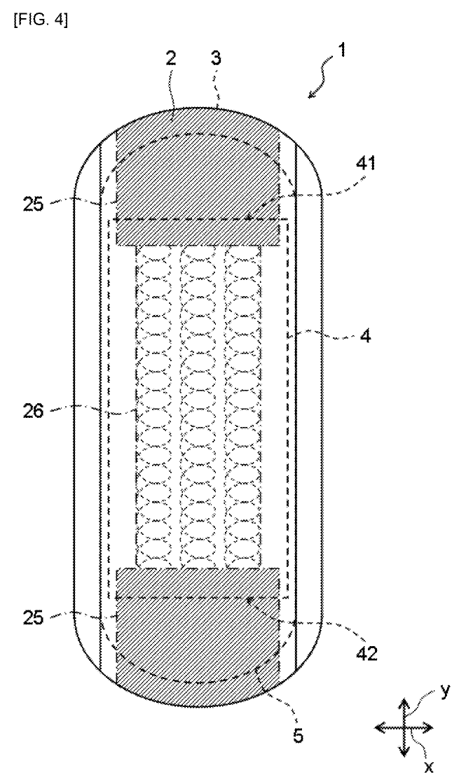

Referring to FIG. 4, a preferred embodiment of arrangement of adhesive coating on the non-skin-facing surface 24 of the top sheet 2 is described. FIG. 4 shows a plan view of the absorbent article 1 with the side sheets 7 removed, as shown in FIG. 1, and shows an example of an adhesive coating arrangement on the non-skin-facing surface 24 of the top sheet 2.

It is preferable that the non-skin-facing surface 24 of the top sheet 2 has a full-surface adhesive coating region 25, over that adhesive is entirely applied and that is arranged at least forward of the front edge 41 of the absorbent body 4 and arranged at least in the portion rearward of the rear edge 42 of the absorbent body 4. With this configuration, the shape retention of the absorbent article 1 tends to be enhanced in the planar direction at a front end portion and a rear end portion of the absorbent article 1. Accordingly, it is easier for a user to grasp the front and rear end portions of the absorbent article 1, and the absorbent article 1 tends to be maintained in a flat state while it is grasped at the front and rear end portions, resulting in better handleability of the absorbent article 1.

The full-surface adhesive coating region 25 over that adhesive is entirely applied is preferably arranged to overlap with the front edge 41 and/or the rear edge 42 of the absorbent body 4 by two to 20 mm inward in the front-rear direction y. For example, in the case where two or more sheet-shaped absorbent layers are provided as the absorbent body 4, when the full-surface adhesive coating region 25 overlaps with the front edge 41 and/or the rear edge 42 of the absorbent body 4 by at least five mm inward in the front-rear direction y, movement of the absorbent polymer in the front and/or the rear direction is suppressed by the full-surface adhesive coating region 25 even if the absorbent polymer swells during use of the absorbent article 1. As a result, it becomes easier to prevent leakage forward and/or backward.

Meanwhile, the non-skin-facing surface 24 of the top sheet 2 preferably has a region that overlaps with the absorbent body and that is not a full-surface adhesive coating region, and the region is more preferably a curved-pattern adhesive coating region 26, in which the adhesive is arranged to extend in a spiral or a meandering pattern in the front-rear direction y. With this configuration, liquid permeability of the top sheet 2 to urine and the like in the region that overlaps with the absorbent body 4 is enhanced. The curved-pattern adhesive coating region 26 is defined as a region that surrounds an adhesive part where the adhesive is applied in a spiral pattern or a meandering pattern.

On the non-skin-facing surface 24 of the top sheet 2, the curved-pattern adhesive coating region 26 is provided at least in a part of the region where the top sheet 2 overlaps with the absorbent body 4, and may also be provided in a portion that does not overlap with the absorbent body 4. It is preferable that the curved-pattern adhesive coating region 26 is provided over a larger area in the region overlapping with the absorbent body 4. In the region overlapping with the absorbent body 4, the length of the curved-pattern adhesive coating region 26 in the front-rear direction y is preferably 60% or longer, more preferably 70% or longer, even more preferably 80% or longer of the length of the absorbent body 4 in the front-rear direction y.

In the curved-pattern adhesive coating region 26, an adhesive part applied in a spiral pattern or a meandering pattern only needs to be formed to extend in the front-rear direction y as a whole. That is, the direction of extension of the repeating pattern of spiral curves or meandering curves need only substantially coincide with the front-rear direction y. The adhesive part means a part where adhesive is actually arranged, and in the curved-pattern adhesive coating region 26, the adhesive part is formed to extend spirally or meanderingly in the front-rear direction y. The adhesive part of a spiral pattern can be formed, for example, by applying adhesive using a spiral coating method, while the adhesive part of a meandering pattern can be formed, for example, by applying adhesive using an omega coating method. The meandering pattern includes, for example, a sinusoidal pattern, a repeating omega-shaped pattern, and the like. Two or more rows of adhesive parts extending meanderingly or spirally in the front-rear direction y are preferably arranged in the width direction x in the curved pattern adhesive coating region.

The full-surface adhesive coating region 25 may be arranged on the non-skin-facing surface 24 of the top sheet 2 in at least a part of the region that is forward of the front edge 41 of the absorbent body 4 and at least a part of the region that is rearward of the rear edge 42 of the absorbent body 4, and is preferably further arranged in a portion overlapping with a front end part and/or a rear end part of the absorbent body 4. More preferably, the full-surface adhesive coating region 25 on the non-skin-facing surface 24 of the top sheet 2 is arranged from the portion overlapping with the absorbent body 4 to the region that does not overlap with the absorbent body 4. That is, the full-surface adhesive coating region 25 on the non-skin-facing surface 24 of the top sheet 2 is preferably arranged to the portion that is forward of the front edge 41 of the absorbent body 4 and to the portion that is rearward of the rear edge 42 of the absorbent body 4. With this configuration, the absorbent article 1 becomes less likely to bend unintentionally at the front edge 41 and the rear edge 42 of the absorbent body 4 when a user handles the absorbent article 1 by grasping the front and rear end portions of the absorbent article 1 where the absorbent body 4 is not present.

In the case that the full-surface adhesive coating region 25 is arranged as described above, the length in the front-rear direction y of an overlapping portion, where the full-surface adhesive coating region 25 overlaps with at least one of the front end part and the rear end part of the absorbent body 4 is preferably 20% or shorter of the length in the front-rear direction y of the absorbent body 4. The full-surface adhesive coating region 25 may have an overlapping portion that overlaps with the front end part of the absorbent body, and the length in the front-rear direction y of the overlapping portion may be 20% or shorter of the length in the front-rear direction y of the absorbent body 4. Also, the full-surface adhesive coating region 25 may have an overlapping portion that overlaps with the rear end part of the absorbent body 4, and the length in the front-rear direction y of the overlapping portion may be 20% or shorter of the length in the front-rear direction y of the absorbent body 4. Furthermore, the full-surface adhesive coating region 25 may have overlapping portions that overlap with the front end part and the rear end part of the absorbent body 4, and the total length in the front-rear direction y of the overlapping portions may be 20% or shorter of the length in the front-rear direction y of the absorbent body 4. Preferably, in the full-surface adhesive coating region 25 that overlaps with the absorbent body 4, the length in the front-rear direction y of the overlapping portion of the full-surface adhesive coating region 25 is 20% or shorter of the length in the front-rear direction y of the absorbent body 4. That is, the total length in the front-rear direction y of the overlapping portion, that overlaps with the absorbent body 4, of the full-surface adhesive coating region 25 is 20% or shorter of the length in the front-rear direction y of the absorbent body 4. Furthermore, it is more preferable that the length in the front-rear direction y of the overlapping portion that overlaps with a front half of the absorbent body 4, of the full-surface adhesive coating region 25 is 10% or shorter in the front-rear direction y of the length of the absorbent body 4, and the length in the front-rear direction y of the overlapping portion that overlaps with a rear half of the absorbent body 4 is 10% or shorter in the front-rear direction y of the length of the absorbent body 4.

The full-surface adhesive coating region 25 on the non-skin-facing surface 24 of the top sheet 2 is preferably arranged over a larger area forward of the front edge 41 of the absorbent body 4. Specifically, in the region forward of the front edge 41 of the absorbent body 4 on the top sheet 2, the length in the front-rear direction y of the full-surface adhesive coating region 25 is preferably 50% or longer, more preferably 70% or longer, even more preferably 80% or longer, and particularly preferably 100% of the length in the front-rear direction y of the region forward of the front edge 41 of the absorbent body 4. That is, it is particularly preferable that the full-surface adhesive coating region 25 extends up to the front edge of the top sheet 2. As the front edge of the top sheet 2 is directly and securely bonded to the front edge of the back sheet 3, it is expected to prevent leakage forward.

The full-surface adhesive coating region 25 on the non-skin-facing surface 24 of top sheet 2 is preferably arranged over a larger area in the portion rearward of the rear edge 42 of the absorbent body 4. Specifically, in the portion rearward of the rear edge 42 of the absorbent body 4 on the top sheet 2, the length of the full-surface adhesive coating region 25 in the front-rear direction y is preferably 50% or longer, more preferably 70% or longer, even more preferably 80% or longer, and particularly preferably 100% of the length of the portion rearward of the rear edge 42 of the absorbent body 4 in the front-rear direction y. That is, it is particularly preferable that the full-surface adhesive coating region 25 extends up to the rear edge of the top sheet 2. As the rear edge of the top sheet 2 is directly and securely bonded to the rear edge of the back sheet 3, it is expected to prevent leakage rearward.

The width of the full-surface adhesive coating region 25 in the width direction x and the width of the curved-pattern adhesive coating region 26 in the width direction x are not particularly limited, but it is preferable that the length of the full-surface adhesive coating region 25 in the width direction x is longer than the length of the curved-pattern adhesive coating region 26 in the width direction x. This makes it easier for a user to grasp the front and rear end portions of the absorbent article 1 and to handle it, and also makes it easier to maintain the absorbent article 1 in a flat state while grasping the front and rear end portions of the absorbent article 1. Therefore, the handleability of the absorbent article 1 when grasped by the front and rear end portions is improved.

The width of the full-surface adhesive coating region 25 in the width direction x is preferably 70% or wider, more preferably 80% or wider of the width of the top sheet 2 in the width direction x. Furthermore, the width of the full-surface adhesive coating region 25 in the width direction x is preferably 40% or wider, more preferably 45% or wider of the length of the absorbent body 4 in the width direction x.

The width of the curved-pattern adhesive coating region 26 in the width direction x is preferably 50% or wider, more preferably 60% or wider of the length of the top sheet 2 in the width direction x. Furthermore, the width of the curved-pattern adhesive coating region 26 in the width direction x is preferably 30% or wider, more preferably 35% or wider, even more preferably 70% or wider of the width of the absorbent body 4 in the width direction x. In order to maintain the shape of the absorbent article 1, the curved-pattern adhesive coating region 26 may be disposed to be up to 110% of width of the absorbent body 4 in the width direction x.

In each adhesive region on the non-skin-facing surface 24 of the top sheet 2 described above, the top sheet 2 is adhered to a member that contacts on its non-skin-facing side. In the portion of the top sheet 2 overlapping with the absorbent body 4, the top sheet 2 may be adhered to the absorbent body 4, or a second sheet may be placed between the top sheet 2 and the absorbent body 4 and the top sheet 2 may be adhered to the second sheet. In the portion of the top sheet 2 not overlapping with the absorbent body 4, the top sheet 2 may be adhered to the back sheet 3, or a base sheet 5 may be placed between the back sheet 3 and the absorbent body 4, and the top sheet 2 may be adhered to the base sheet 5. In the portion of the top sheet 2 overlapping with the absorbent body 4, it is preferable that the top sheet 2 is directly adhered to the absorbent body 4. With this configuration, the permeability of urine and the like can be improved in the curved-pattern adhesive coating region 26.

The top sheet 2 formed as described above is particularly preferably applied when the absorbent body 4 is a sheet-shaped absorbent body. The sheet-shaped absorbent body is configured to contain an absorbent polymer but not contain a pulp fiber between sheet members, and since the absorbent article 1 provided with the sheet-shaped absorbent body is formed thin and has a relatively large difference in shape retention between the portion where the absorbent body 4 is present and the portion where it is not present, the absorbent article 1 is prone to bending at the front edge 41 and the rear edge 42 of the absorbent body 4. However, by providing the top sheet 2 formed as described above, the absorbent article 1 is less likely to bend unintentionally at the front edge 41 and the rear edge 42 of the absorbent body 4, and the handleability of the absorbent article 1 can be improved.

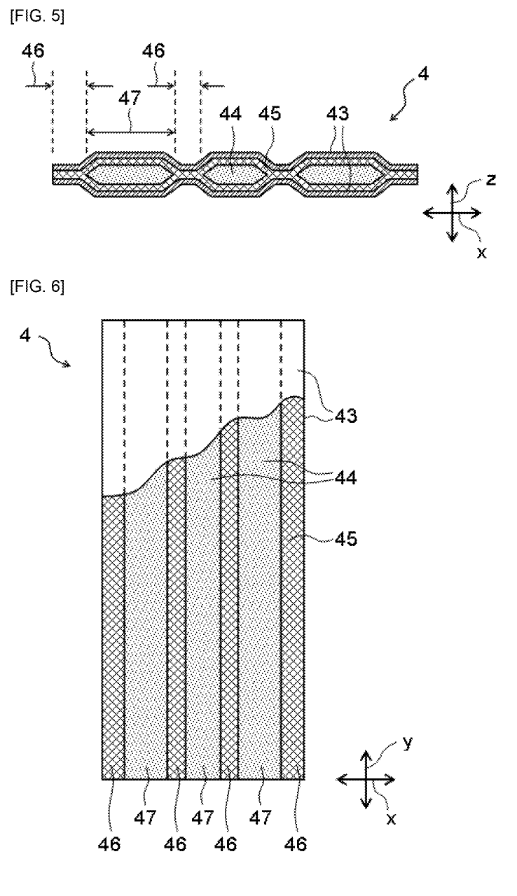

An example of the configuration when the absorbent body 4 is a sheet-shaped absorbent body is described with reference to FIGS. 5 and 6. FIG. 5 shows a cross-sectional view in the width direction x of the absorbent body 4 of a sheet-shaped absorbent body, and FIG. 6 shows a partially cutaway plan view of the absorbent body 4 of the sheet-shaped absorbent body as seen from the top sheet 2 side.

The absorbent body 4 includes two or more sheet members 43, an absorbent polymer 44 without a pulp fiber is disposed between the sheet members 43. That is, in the absorbent body 4, one of the sheet members 43 is disposed at a side closer to a skin-facing surface of the absorbent body 4 and the other of the sheet members 43 is disposed at a side closer to a non-skin-facing surface of the absorbent body 4, and the absorbent polymer 44 is disposed between the sheet members 43, but a pulp fiber is not disposed between the sheet members 43. It is noted that the presence of a pulp fiber inevitably mixed in between the sheet members 43 during manufacturing is allowed. With the configuration of the absorbent body 4 above, the absorbent body 4 can be thinned while having a sufficient absorption capacity.

The shape (planar shape) of the absorbent body 4 is not particularly limited. Examples of the shape of the absorbent body 4 include a substantially rectangular shape, an hourglass shape, a gourd shape, a battledore shape and others.

As the absorbent polymer 44, a polyacrylic absorbent polymer such as sodium polyacrylate; a starch-based absorbent polymer such as starch-acrylonitrile graft copolymer, starch-acrylic acid graft copolymer and starch-acrylamide graft copolymer; a polyvinyl alcohol absorbent polymer such as crosslinked polyvinyl alcohol; or the like can be used. As the absorbent polymer 44, a polyacrylic absorbent polymer such as sodium polyacrylate is preferably used, in view of high liquid absorption ability.

The sheet member 43 is liquid permeable, and a nonwoven fabric made from hydrophilic fibers such as cellulose, rayon and cotton; a nonwoven fabric which is formed from hydrophobic fibers such as polyolefin (e.g., polypropylene and polyethylene), polyester (e.g., PET) and polyamide (e.g., nylon), and in which the hydrophobic fibers are hydrophilized with a surfactant on the surface thereof; or the like can be used, for example. As the sheet member 43, a woven fabric, a knitted fabric, a plastic film having holes or the like may be also used. Preferably, a nonwoven fabric sheet member, namely, a nonwoven fabric sheet is used, as the sheet member 43.

For the sheet member 43, a first sheet member may be disposed at a side closer to the skin-facing surface of the absorbent article and a second sheet member may be disposed at a side closer to the non-skin-facing surface of the absorbent article, or a single sheet member may be folded back, and one side of the folded single sheet member may serve as the first sheet member at a side closer to the skin-facing surface and the other side of the folded single sheet member may serve as the second sheet member at a side closer to the non-skin-facing surface of the absorbent article.

The absorbent body 4 includes a sealed part 46 in which the sheet members 43 are bonded to each other and a non-sealed part 47 in which the sheet members 43 are not bonded to each other, and the absorbent polymer 44 is disposed in the non-sealed part 47. The sealed part 46 is formed by bonding the sheet members 43 to each other with an adhesive or by welding (heat sealing, ultrasonic welding, or the like). The non-sealed part 47 is defined as a part where the sheet members 43 are not bonded to each other, and a part other than the sealed part 46 corresponds to the non-sealed part 47. As an absorbent body 4, an absorbent polymer particle may also be present in the sealed part 46.

In FIG. 5, adhesive is applied to the sheet member 43 to form an adhesive layer 45, and the absorbent polymer 44 is bonded to the sheet member 43 by the adhesive layer 45 in the non-sealed part 47. The adhesive layer 45 may be provided on an inner surface of at least one of the sheet member 43 that is positioned at a side closer to the skin-facing surface and the sheet member 43 that is positioned at a side closer to the non-skin-facing surface. The adhesive layer 45 is preferably provided on inner surfaces of both of the sheet members 43 positioned at the side closer to the skin-facing surface and positioned at the side closer to the non-skin-facing surface. At least a part of the absorbent polymer 44 may be bonded to the adhesive layer 45. When the absorbent polymer 44 is bonded to the sheet member 43 by the adhesive layer 45, particles of the absorbent polymer 44 hardly move between the sheet members 43 prior to absorption of the absorbent polymer 44, and the ability of the absorbent body 4 to absorb urine and the like is easily secured. Furthermore, even after the absorbent polymer 44 has absorbed liquid, gelled absorbent polymer 44 would be less likely to move between the sheet members 43, and as a result, it hardly occurs to give a wearer uncomfortable feeling due to the lump-formed absorbent polymer 44.

It is preferable that the adhesive layer 45 does not inhibit water absorption or swelling of the absorbent polymer 44 while bonding the absorbent polymer 44 to the sheet members. In order to form such an adhesive layer 45, the adhesive layer 45 is preferably formed by a curtain spray method, a spiral coating method, an omega coating method, a coater method or the like.

The sealed part 46 preferably maintains the bonding between the sheet members 43 even after absorption of the absorbent polymer 44. With this configuration, the ability of the absorbent body 4 to absorb urine and the like tends to be secured even after initial liquid absorption of the absorbent polymer 44. From this standpoint of view, the sealed part 46 is preferably formed by bonding the sheet members 43 to each other with an adhesive such as a rubber-based adhesive or a styrene-based elastomer or formed by welding the sheet members 43 to each other.

In the absorbent body 4, the non-sealed parts 47 that extend in parallel in the front-rear direction y are arranged in a stripe pattern. That is, at least two non-sealed parts 47 are arranged to extend in the front-rear direction y. In this case, at least one sealed part 46 that extends in the front-rear direction y is arranged between the non-sealed parts 47 in the width direction x. Preferably, a first sealed part of the sealed parts 46 extending in the front-rear direction y is arranged at a side closer to one end of the absorbent body 4 in the width direction x and a second sealed part of the sealed parts 46 extending in the front-rear direction y is arranged at a side closer to the other end of the absorbent body 4 in the width direction x. Therefore, it is preferable that the sealed parts 46 that extend in the front-rear direction y are also arranged in a stripe pattern. The sealed parts 46 are preferably arranged to extend over the entire length in the front-rear direction y. With this configuration of the absorbent body 4, urine and the like tends to be easily diffused in the front-rear direction y at the sealed parts 46, and also, urine and the like reaching the absorbent body 4 through the top sheet 2 tends to be further diffused and absorbed rapidly in the absorbent body 4. Three or more non-sealed parts 47 are preferably arranged side by side in the width direction x. Four or more sealed parts 46 are preferably arranged side by side in the width direction x.

The width of each of the sealed parts 46 in the width direction x is preferably 1 mm or wider, more preferably 2 mm or wider, and preferably 10 mm or narrower. The width in the width direction x of each of the sealed parts 46 that are located at the outermost positions in the width direction x is preferably wider than the width in the width direction x of each of the sealed parts 46 that are located inward of the sealed parts 46 located at the outermost position in the width direction x. The width of each of the sealed parts 46 that are located at the outermost position in the width direction x is preferably 2 mm or wider and 10 mm or narrower. The width in the width direction x of each of the sealed parts 46 that are located inward in the width direction x is preferably narrower than the width of the sealed parts 46 that are located at the outermost positions in the width direction x, and is preferably 1 mm or wider and 5 mm or narrower. With the above configuration, when the absorbent polymer 44 expands during use of the absorbent article 1, movement and expansion of the absorbent polymer 44 in the width direction x is moderately suppressed without hindering its swelling.

The width of each of the non-sealed part 47 in the width direction x is preferably 5 mm or wider, more preferably 8 mm or wider, and preferably 50 mm or narrower, more preferably 30 mm or narrower, even more preferably 20 mm or narrower. The width in the width direction x of each of the non-sealed parts 47 located at the outermost positions in the width direction x is preferably wider than the width in the width direction x of each of the non-sealed part 47 located inward of the non-sealed parts 47 located at the outermost positions in the width direction x.

The amount of the absorbent polymer 44 disposed in the non-sealed parts 47 of an absorbent layer is not particularly limited, and is preferably 100 g/m² or more, more preferably 150 g/m² or more, and preferably 400 g/m² or less. When the absorbent polymer 44 is disposed in the non-sealed parts 47 of the absorbent layer in the amount of 100 g/m² or more, absorption capacity of the absorbent body 4 can be easily increased. Meanwhile, when the absorbent polymer 44 is disposed in the non-sealed parts 47 of the absorbent layer in the amount of 400 g/m² or less, the bonding between the sheet members 43 in the sealed parts 46 is easily maintained even when the absorbent polymer 44 absorbs liquid.

### REFERENCE SIGNS LIST

1: absorbent article

2: top sheet

21: thick part

22: thin part

23: skin-facing surface

24: non-skin-facing surface

25: full-surface adhesive coating region

26: curved-pattern adhesive coating region

3: back sheet

4: absorbent body

41: front edge

42: rear edge

43: sheet member

44: absorbent polymer

45: adhesive layer

46: sealed part

47: non-sealed part

5: base sheet

6: standing flap

7: side sheet

8: elastic member for standing flap

1. An absorbent article having a front-rear direction and a width direction, comprising a top sheet, a back sheet and an absorbent body disposed between the top sheet and the back sheet:
the top sheet comprising thick parts and thin parts that extend in parallel in the front-rear direction and are arranged alternately in the width direction;
the thick parts and the thin parts extending from a portion overlapping with the absorbent body to positions forward beyond a front edge of the absorbent body and to positions rearward beyond a rear edge of the absorbent body;
the top sheet having a skin-facing surface with the thin parts that are recessed relative to the thick parts and a non-skin-facing surface with the thin parts that are recessed relative to the thick parts; and
the thickness of the thick part being 3 times or more and 15 times or less the thickness of the thin part.

2. The absorbent article according to claim 1, wherein
the top sheet is a nonwoven fabric that is heat-embossed at the thin parts.

3. The absorbent article according to claim 2, wherein
the nonwoven fabric comprises a fused portion of fibers, in a vicinity of a boundary between the thick part and the thin part,
a ratio of a long fused portion, in which a fusion length between fibers is 10 times or more a diameter of the fiber, is less than 5%, and
a total ratio of a medium fused portion, in which the fusion length of fibers is 3 times or more and less than 10 times the diameter of the fiber, and a short fused portion, in which the fusion length of fibers is 1.5 times or more and less than 3 times the diameter of the fiber, is 20% or more and 100% or less.

4. The absorbent article according to claim 1, wherein
the mass per unit area of the top sheet is 20 g/m² or more and 35 g/m² or less.

5. The absorbent article according to claim 1, wherein
the thick parts and the thin parts extend continuously in the front-rear direction from a front edge to a rear edge of the top sheet.

6. The absorbent article according to claim 1, wherein
the front edge of the absorbent body is straight and the rear edge of the absorbent body is straight.

7. The absorbent article according to claim 1, wherein
the top sheet comprises a portion that is positioned forward of the front edge of the absorbent body and the portion has a length, that is 2 cm or longer and 7 cm or shorter, in the front-rear direction, and
the top sheet comprises a portion that is positioned rearward of the rear edge of the absorbent body and the portion has a length, that is 2 cm or longer and 7 cm or shorter, in the front-rear direction.
